(19) United States
(12) Patent Application Publication
(10) Pub. No.: US 2021/0260397 A1
(43) Pub. Date: Aug. 26, 2021

(54) SKIN TREATMENT

(71) Applicants: Steve Mencanin, Lake Forest, CA (US); Shienlin Saber, Laguna Beach, CA (US)

(72) Inventors: Steve Mencanin, Lake Forest, CA (US); Shienlin Saber, Laguna Beach, CA (US)

(21) Appl. No.: 16/418,963

(22) Filed: May 21, 2019

Related U.S. Application Data
(60) Provisional application No. 62/675,094, filed on May 22, 2018.

(51) Int. Cl. A61N 5/06; A61K 41/00; A61K 31/80

(52) U.S. Cl. CPC A61N 5/062; A61K 41/0057; A61N 2005/0659; A61N 2005/0651; A61K 31/80

(57) ABSTRACT

The present invention relates to methods and uses of combined electromagnetic radiation and a polymerizable composition for the treatment of skin lesions, including sores, herpetic sores, ulcerations and wounds.

The present invention relates to the use of electromagnetic radiation in combination with a polymerizable composition for the treatment of skin lesions, sores and wounds, especially for herpes virus infections and ulcers. The invention includes inter a/ia kits for and methods of treatment of herpetic infections and other skin lesions.

### BACKGROUND

There are at least eight herpesvirus types that infect humans: herpes simplex viruses 1 and 2, varicella-zoster virus, EBV (Epstein-Barr virus), human cytomegalovirus, human herpesvirus 6, human herpesvirus 7, and Kaposi's sarcoma-associated herpesvirus, all of which are extremely widespread among humans. More than 90% of adults have been infected with at least one of these, and a latent form of the virus remains in most people.

Herpes simplex viruses are categorized into two main types: herpes type 1 (HSV-1, or oral herpes) and herpes type 2 (HSV-2, or genital herpes). Most commonly, herpes type 1 infection causes sores around the mouth and lips, although HSV-1 can cause genital herpes, most cases of genital herpes infection are caused by herpes type 2. Herpes infections are painful and can be unsightly causing emotional distress, especially when they present as multiple facial lesions. For many people with the herpes virus, the virus can go through periods of being dormant, attacks (or outbreaks) can be brought on by the following conditions: general illness (from mild illnesses to serious conditions), fatigue, physical or emotional stress, immunosuppression due to AIDS or such medications as chemotherapy or steroids, trauma to the affected area, including sexual activity and menstruation. Furthermore, it is known that UV B light (a natural component of sunlight) is a potent stimulus for inducing reactivation of latent herpes simplex virus (HSV) infections. When an individual is first infected with herpes and exhibits active symptoms, the duration of the primary outbreak will be the worst and most painful and can take 3 to 6 weeks to resolve. Subsequent recurrent outbreaks will be less severe, typically lasting up to 14 days and the frequency will become farther and farther apart with each recurring attack.

It is known from the prior art to treat herpetic infections by phototherapy or by administration of oral or topical antiviral agents. For example, electromagnetic radiation centred around −1072 nm can be used for the reduction in duration of herpes virus infections (WO 9919024). Furthermore, it has been shown that electromagnetic radiation centred around 1072 nm can reduce the duration of a herpetic infection (4.6+i−2.2 days) as compared to the conventional antiviral Aciclovir™ topical treatment (8.i+/−2.5 days).

Despite the recent improvements in herpes virus treatments, there remains a need for new herpetic treatments that can further reduce infection duration and to increase the period between recurrence of a subsequent infection.

There is also a need for improved treatments for ulcers such as diabetic ulcers and pressure sores.

### SUMMARY OF THE DISCLOSURE

According to a first aspect of the invention there is provided use of electromagnetic radiation centred around 1072 nm in combination with a polymerizable composition for the treatment of skin conditions selected from the group comprising herpes virus infections, ulcerations and wounds.

Preferably, the electromagnetic radiation is divergent light between 10° to 50°. By divergent it is meant that the electromagnetic radiation emitted from the electromagnetic source has a divergent half angle of at least 5°. Preferably divergence of the electromagnetic radiation is in the range 15° to 25° half angled divergent. Thus it will be appreciated that the method of the present invention does not include the use of lasers as the source of the electromagnetic radiation.

It has now been surprisingly established that low intensity electromagnetic radiation of small bandwidth (preferably around 10 nm to 120 nm and more preferably 50 nm), in combination with a polymerizable composition, is more effective in reducing the duration of a herpetic infection that the light on its own. It is postulated that the way in which the electromagnetic radiation effects its action is by way of energy transmission through cellular components/organelles, enzymes such as but not limited to inducible nitric oxide synthase (iNOS). A water molecule that has a range of electromagnetic radiation wavelengths passed through it will produce several transmission peaks. These transmission peaks are associated with the preferred therapeutic electromagnetic radiation wavelength range of the invention and thus implies a role for the water molecule in the general mechanism of action. The polymerizable composition permits transmission of the electromagnetic radiation whilst at the same time providing a seal over the affected herpetic area. The light and the seal provide a combined improvement therapeutic effect.

Studies have shown that wavelengths centred around those wavelengths specified above and especially around single restricted bandwidth light centred at 1072 nm or 1267 nm are particularly effective. It is of note that these two wavelengths correspond to the peak emission wavelengths of a water molecule light transmission profile and thus we believe that the mechanism of action is related to water and possibly cell membranes. Preferably, the electromagnetic radiation is continuous or pulsed.

Preferably, when the electromagnetic radiation is continuous the intensity is at least 500 μWatts/cm² and up to 500 mWatts/cm². Preferably when the electromagnetic radiation is pulsed the intensity is at least 500 μWatts/cm² peak power and the average power is up to 500 mWatts/cm². The average power is the peak power multiplied by the proportion of the total time that the radiation is applied. For instance, if the peak power is 500 μWatts/cm² and is pulsed for 10 μseconds at a frequency of 600 Hz then the average power is 30 μWatts/cm². Prior art methods which rely on thermal warming specify a lower limit of 0.5 Watts/cm² the present invention which seeks to avoid any thermal effects operates below this level. Preferably when the electromagnetic radiation is pulsed the average power of the intensity is in the region of 50-100 μWatts/cm².

We have found that the power may suitably range from 500 μWatts/cm² peak to 500 mWatts/cm² continuous or peak power when applied to the skin. Typically, 20 mWatts/cm² are used on skin but this value is dependent on how fat or muscular the subject is and thus how deep the wound lies.

Preferably when the electromagnetic radiation is pulsed it is applied for periods of at least 10-15 μseconds and more preferably is applied at a frequency/repetition rate in the range 300-900 Hz more preferably still the frequency/repetition rate is at, or about, 600 Hz.

Our studies have shown that the electromagnetic radiation can be either coherent or non-coherent the clinical outcomes are not affected by this parameter.

Preferably the electromagnetic radiation is applied to the affected area for at least 3 to 20 minutes. A typical exposure time is in the region of 5 to 10 minutes, however for deeper wounds this time is increased according to the individual's fat layer depth and exposure could be up to 15 minutes.

Preferably, the electromagnetic radiation is applied to the affected site at least one a day and over a period of days, until the herpetic sores have ceased to exudate and have formed scabs. It is expected that a treatment protocol would include a treatment of several times per day such as 2, 3 4, 5, 6 times per day.

It should be appreciated that the power source emitting the electromagnetic radiation will have to produce more than the required intensity for the clinical effect since we have shown that approximately 99% of the applied therapeutic amount of light is lost across the skin surface during treatment. Thus the intensity of applied radiation will have to be corrected for when carrying out a treatment.

From the foregoing it is understood that the electromagnetic radiation may be directed to the affected site either continuously or in a switched (pulsed) manner. The main benefit of switching enables power conservation and facilities much higher peak power output, thereby improving the healing response.

Preferably, the electromagnetic radiation therapy source includes means for reducing the amount of ambient radiation, which impinges on the treatment site.

Preferably, the electromagnetic radiation source is a light emitting diode. The radiation from such devices can be electrically operated or the radiation can be delivered to an applicator via a fibre-optic delivery system.

Preferably, the radiation source emitter includes a PN junction arranged to emit radiation with a wavelength centring at or about the previously mentioned specified wavelengths. A single light diode assembly may include a plurality of orientated junctions. Infrared emitting diodes may be arranged not only to emit radiation at a specific frequency but also to emit a high intensity divergent beam. The divergent light may also be derived from light emitting polymers.

The present invention is concerned with a method of treating skin conditions and especially herpetic infections, with divergent electromagnetic radiation having a wavelength centred around 1072 nm and optionally also 1267 nm in combination with a polymerizable composition. The electromagnetic radiation is applied at a low intensity such that no thermal damage or heating is caused to the skin or any other tissue or organ around the treatment area. In this way, the method of the present invention differs from the prior art as the effects are non-thermal and avoid thermolysis. The electromagnetic radiation passes through the polymerizable composition which forms a flexible or elastic seal over the affected area.

Preferred materials include biocompatible materials that have pliability in hardened state such that movement of the body and/or affected surface will be accommodated by corresponding elastic deformation (without dislodgment from the affected area). Thus, especially preferred sealing materials include various polymerizable compositions that can polymerize in a relatively short period (e.g., less than 10 min, less than 5 min, less than 2 min, less than 1 min, etc.). Preferably, polymerizable composition polymerizes within 2 minutes of contact with the skin.

Preferably, the silicone mixture comprises any one or more of the following components selected from the group comprising a siloxane polymer, a fumed amorphous silica, a platinum catalyst, a methyl hydrogen crosslinker, an inhibitor, a thixotropic agent optionally wherein the thixotropic agent is polydimethyl siloxane and an adhesion promoter optionally wherein the adhesion promoter is tetrapropoxysilane.

Preferably, the silicone mixture comprises:
(i) a first silicone elastomer component comprising a first platinum catalyst; and
(ii) a second silicone elastomer component comprising a silicone crosslinker.

Preferably, the silicone mixture has a work time of between 30-90 seconds, and a cure time of between 1-5 minutes.

Preferably, the seal has a hardness of between 0 on the Shore 00 durometer scale and 40 on the Shore A durometer scale.

Preferably, the seal has a hardness of between 15-25 on the Shore A durometer scale. Preferably, the seal has an elongation at break of between 200-800%.

Preferably, the seal has an elongation at break of between 400-650%. Preferably, the seal has a tensile strength of between 100-2000 psi.

Preferably, the seal has a tensile strength of between 200-800 psi.

Preferably, wherein prior to application of the polymerizable composition there is provided use of a base component in order to increase adhesion and/or improve cure time of the polymerizable composition.

Preferably, the base component is applied as a spray, foam or wipe.

Preferably, the base component comprises a catalyst that accelerates polymerization of the polymerizable composition.

Preferably, the composition further includes any one or more of the following components selected from the group comprising: an inert pigment; a flesh tone pigment, an aesthetic; an anti-inflammatory; a camouflaging agent; an antiviral; an antibiotic and an analgesic.

A particular advantage of the polymerizable composition of the present invention is that it forms an elastic seal over the affected site and upon healing of the site can be peeled off without damage to the underlying epithelia of tissue. Indeed, a further advantage of the polymerizable compositions is that upon application and polymerization, scab or crust formation is substantially prevented, which significantly helps reduce inadvertent re-injury by accidental dislodgment of the crust or scab.

According to a further aspect of the invention there is provided a method of treatment of a skin lesion, comprising:

applying a polymerizable composition to the affected area of skin;

wherein the polymerizable composition is formulated to cure to a hardness within 5 minutes;

allowing the polymerizable composition to harden to the hardness of between 0 on the Shore 00 durometer scale and 40 on the Shore A durometer scale to so form an elastic protective barrier; and

applying electromagnetic radiation centred around 1072 nm over the cured composition.

Preferably, the skin lesion is selected from the group comprising herpetic sores, diabetic ulcers, pressure sores and wound.

As mentioned hereinbefore the polymerizable composition naturally forms a contoured seal over the lesion taking the shape around the lesion and into any crevices, pits of folds of skin. It is also easily peeled from the lesion following treatment.

According to a yet further aspect of the invention there is provided a kit for treating a skin lesion, comprising:
(i) a base component comprising at least one of an adhesion promoter, a catalyst that accelerates polymerization of the polymerizable composition;
(ii) a polymerizable composition formulated to polymerize to form a removable seal; and
(iii) a device for emitting electromagnetic radiation centred around 1072 nm.

Preferably, the kit includes any one or features hereinbefore described.

It will be appreciated that each and every feature ascribed to one aspect of the invention applies mutatis mutandis to each any every other aspect of the invention.

### BRIEF DESCRIPTION OF THE DRAWINGS

Embodiments of the invention are further described hereinafter with reference to the accompanying drawings, in which:

FIG. 1 shows Treatment of herpetic lesion by 1072 nm.

FIG. 2 Shows Treatment with polymer after 1072 nm treatment

FIG. 3 Shows healed lesion after treatment of 1072 nm and polymer

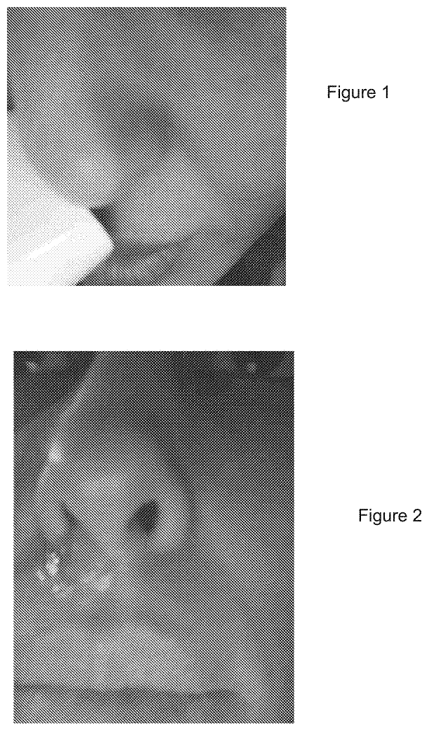

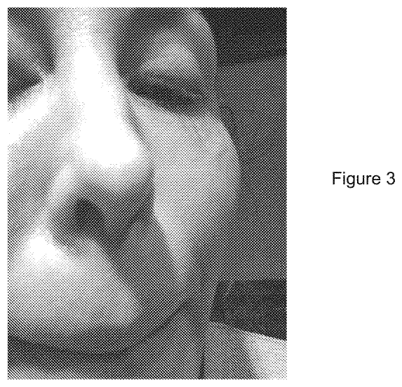

### DETAILED DESCRIPTION

Reference hereinto “treatment” is intended to be considered in its broadest context and does not necessarily imply that a subject is treated until total recovery. It includes reducing the duration, severity or pain associated with a skin sore, amelioration of symptoms spreading of a herpetic infection.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

One type of a preferred polymerizable composition comprises a two-part silicone elastomer system that cures at room (20 degree Celsius) or body (37 degree Celsius) temperature and includes (a) a first formulation including a polymer and catalyst (e.g., silicone polymer and platinum or other suitable metal catalyst), and (b) a second formulation comprising a polymer and a crosslinker. One or both of the formulations could include one or more of a filler, a thixotropic agent, an adhesion promoter, and a cure inhibitor to control the cure kinetics.

In some contemplated embodiments, the polymer is a silicone polymer (e.g., siloxane polymer) with a polymer backbone of alternating silicone and oxygen atoms (i.e., siloxane bonds), and hydrocarbon (saturated, unsaturated, aromatic) organic side groups such as methyl, phenyl or vinyl, or a hydrogen attached to the silicon atoms. The siloxane polymer can comprise between 20-100 wt %, more preferably at least 50 wt %, and even more preferably at least 70 wt % (e.g., between 75-85 wt %, between 78-82 wt %) of the polymerizable composition (i.e., of the combined two part formulation where the catalyst and crosslinker are combined).

For example, where polydimethylsiloxane (PDMS) is used, it can be a linear polymer made up of repeating Si—O—Si linkages and a reactive vinyl group on both ends of the polymer chain.

There may be organic side groups such as dimethyl bonded to every silicone molecule the backbone of the polymer. Siloxane polymers can also be substituted with diphenyl, methylphenyl, trifluoropropyl, or any combination thereof. Some exemplary siloxanes include oligosiloxanes, polydimethylsiloxane (PDMS), vinyl-endblocked polydiphenyl siloxane, vinyl-endblocked polymethylphenylsiloxane, vinyl-endblocked trifluoropropyl siloxane, vinyl-endblocked polydiethyl siloxane, trimethyl-endblocked methylvinyl polydimethylsiloxane, trimethyl-endblocked methylvinyl polydiphenylsiloxane, trimethyl-endblocked methylvinyl polymethylphenylsiloxane, trimethyl-endblocked methylvinyl polytrifluoropropylsiloxane, and trimethyl-endblocked ethylvinyl polydimethylsiloxane. Contemplated siloxanes can be optically clear, non-toxic and non-flammable.

All suitable chain lengths of the siloxane polymer are contemplated, including between 10-2,500 repeating units long, between 200-1,000 repeating units long, or between 200-400 repeating units long, or between 300-600 repeating units long, or between 300-400 repeating units long (e.g., 340-360), or between 500-800 repeating units long, or between 700-1,000 repeating units long. Thus, siloxane polymers may have an average molecular weight of between about 500-5,000 Daltons, or between about 5,000-20,000 Daltons, or between about 15,000-35,000 Daltons, or between about 35,000-55,000 Daltons, or between about 55,000-100,000 Daltons, or more.

According to another embodiment, a polymer can include a main chain formed primarily of organosiloxane units. Among the silicone compounds contemplated, some may display both curing and adhesive properties, for example depending on the proportion of silicone or whether they are used with a particular additive. It may therefore be possible to adjust the properties of said compositions according to the proposed use.

In some contemplated embodiments where the polymer is a siloxane, the crosslinker is a siloxane crosslinker such as a methyl-hydrogen crosslinker. The crosslinker can comprise between 0.1-50 wt %, between 0.1-10 wt %, and more preferably between 1-5 wt % (e.g., 2 wt %) of the polymerizable composition. An exemplary siloxane crosslinker used in some contemplated compositions is a small chain polymer that is trimethyl endblocked, making the ends of the chain non-functional. All suitable chain lengths of the crosslinker are contemplated, including for example, between 1-100 repeating units, more preferably between 1-50 units, and more preferably between 5-15 units (e.g., 10 units wherein the molecular weight is 800 Daltons).

Along the backbone of the crosslinker can be reactive methyl-hydrogen side groups which can comprise between 1-99 mole %, more preferably between 20-80 mole %, and more preferably between 40-60 mole % (e.g., 50 mole %) of the crosslinker. The remaining mole % can comprise dimethyl side groups. Where each of the methyl-hydrogen side groups and the dimethyl side groups make up approximately 50 mole %, approximately half of the repeating units of the crosslinker will be dimethyl, and approximately half will be methyl hydrogen. Other contemplated crosslinkers include hydride-endblocked polydimethylsiloxane, hydride-endblocked methylhydrogen polysiloxane, trimethyl-endblocked methylhydrogen methylvinyl polysiloxane, trimethyl-endblocked 100 mole % methylhydrogen polysiloxane, hydride-endblocked polydiphenylsiloxane, and hydride-endblocked phenylhydrogen polysiloxane. Although the exemplary crosslinkers described above are siloxane crosslinkers, it should be appreciated that a person skilled in the art would be able to select a suitable crosslinker based on the polymer included in the polymerizable compositions.

The catalysts of contemplated polymerizable formulations can comprise a peroxide, platinum, tin, a combination thereof, or other suitable catalyst. An exemplary platinum catalyst for hydrosilylation reactions can comprise a complex of platinum with a vinyl siloxane acting as a ligand. An example of this is the Karstedt's catalyst. Other contemplated catalysts include, rhodium complex in vinyl silicone fluid, organotin catalyst such as dibutyltin dilaurate, stannous octoate, dibutlytin diacetate, peroxide catalysts such as benzoyl peroxide, 2,4 dichlorobenzoyl peroxide, dicumyl peroxide, 2,5-dimethyl-2,5-di(tert-butylperoxy)hexane. In still further contemplated aspects, platinum may be replaced at least to some degree by other metals, including silver and copper, in nanoparticulate form or ionic form, which advantageously will also provide antimicrobial effect.

The catalyst can be present in the formulation in any suitable amount, for example, between 0.001-10 wt % (of the combined two part formulation where the catalyst and crosslinker are combined), more preferably between 0.01 and 1 wt %, and more preferably between 0.07 and 0.13 wt % (e.g., 0.1 wt %) of the polymerizable composition, and can include between 1-250 ppm, between 5-70 ppm, more preferably between 15-60 ppm (e.g., 30 ppm) of pure platinum. Notably, the amount of catalyst will affect the working time of the mixed composition, and higher quantities of catalyst will decrease the time required to form a seal. Thus, higher than normal (e.g., as recommended by manufacturer or published in the relevant art) quantities of catalyst are generally preferred.

Suitable adhesion promoters can also be included in the polymerizable composition to increase the bond strength of the adhesive (polymerizable composition or seal) to the substrate (e.g., skin or ear canal) as curing occurs. Tetrapropoxysilane is an exemplary adhesion promoter commonly used in silicone primers. Without wishing to be bound by any particular theory, the applicant contemplates that the reactive silane may form hydrogen or even covalent bonds with the skin tissue, possibly catalyzed by heme, iron, and/or other components in blood where present. The adhesion promoter, when included in the polymerizable composition, can comprise between 0.01-10 wt %, between 0.1 and 5 wt %, and more preferably between 0.4 and 1.2 wt % (e.g., 0.8 wt %) of the polymerizable composition.

Where a filler is included in the polymerizable formulation, an exemplary filler includes amorphous fumed silica having a surface area of between 100-300 m2/gram (e.g., approximately 200 m2/gram). Other contemplated fillers include fumed silica with low surface area (e.g., 100 m2/gram), fumed silica with high surface area (e.g., 400 m2/gram), precipitated silica, diatomaceous earth, titanium dioxide, zinc oxide, barium sulfate, colloidal silica, and boron nitride.

The filler can comprise between 0-80 wt %, more preferably between 5-35 wt % and even more preferably between 10-23 wt % (e.g., 16 wt %) of the combined two part formulation where the catalyst and crosslinker are combined. The surface of the silica can be treated with trimethylsilyl groups so that it is more soluble with the polymer. Further particularly preferred fillers will be suitable to increase attenuation of sound and may therefore include fumed silica, silica hollow spheres, polymeric elements and foams, and/or blowing agents and/or detergents that produce a (silica) foam.

A suitable thixotropic additive (e.g., a compound that reduces the flowability of a material rendering it non-slump) can also be included in some contemplated polymerizable compositions in any suitable amount. For example, the thixotrope can comprise between 0.1-5 wt %, between 0.5-2.5 wt %, and more preferably between 1-2 wt % (e.g., 1.5 wt %) of the combined two part formulation where the catalyst and crosslinker are combined. An exemplary thixotrope included in some contemplated formulations is a hydroxyl endblocked polydimethyl siloxane with a chain length of between 10-20 repeating units (e.g., 15 repeating units with a molecular weight of 1100 Daltons). The hydroxyl groups on the polymer ends can react with the surface hydroxyl groups of the fumed silica causing the silica to become less flowable.

Exemplary Compositions used for covering the lesions were as shown in Tables 1 and 2 below. Part 1 and Part 2 were mixed in equal quantities immediately prior to application, and a thin film was applied directly to the lesion. After about 2 minutes, the formulations was polymerized and electromagnetic radiation having a wavelength centred around 1072 nm and optionally also 1267 nm was applied.

| | Part | | Part |
|---|---|---|---|
| Component | Wt % | Component | Wt % |
| Vinyl endblocked | 60-90 | Vinyl endblocked | 60-90 |
| polydimethyl | | polydimethyl siloxane | |
| siloxane polymer | | polymer (100-1000 | |
| (100-1000 DP) | | repeating siloxy units) | |
| Fumed silica with | 10-30 | Fumed silica with | 10-30 |
| surface area of 200 | | surface | |
| m</gram (100-400 | | area of 200 m</gram | |
| m</gram) | | | |
| Platinum catalyst | 0.06-0.2 | Trimethyl endblocked | 2-10 |
| complex | | methyl- hydrogen | |
| | | siloxane polymer | |
| | | crosslinker (containing | |
| | | 50% methyl hydrogen | |
| | | and 50% dimethyl) | |
| | | 1.3.5.7-tetramethyl- | .001-.05 |
| | | 1.3.5.7-tetravinyl- | |
| | | cyclotetrasiloxane | |
| | | Tetraprop | 1-5 |
| | | oxysilane | |
| | | adhesion | |
| | | promoter | |
| | | N-(triethoxysilylpropyl)-O- | 0.1-2 |
| | | polyethylene oxide | |
| | | urethane adhesion | |
| | | promoter | |
| | | Optional: Docosanol or | |
| | | acyclovir can be included | |
| | | at commonly applied | |
| | | concentrations | |
| Vinyl endblocked | 79.947 | Vinyl endblocked | 74-75 |
| polydimethyl siloxane | | polydimethyl siloxane | |
| polymer (chain length 350 | | polymer (chain length | |
| repeating units) | | 350 repeating units) | |
| Fumed silica with surface | 19.91 | Fumed silica with surface | 18.5-19 |
| area of 200 m²/gram | | area of 200 m²/gram | |
| Platinum catalyst complex | 0.143 | Trimethyl endblocked methyl- | 3.5-4.0 |
| | | hydrogen siloxane polymer | |
| | | crosslinker (containing | |
| | | 50 wt % methyl hydrogen and | |
| | | 50 wt % dimethyl) | |
| | | 1,3,5,7-tetramethyl-1,3,5,7- | 0.013- |
| | | tetravinyl-cyclotetrasiloxane | 0.017 |
| | | Tetraprop oxysilane adhesion | 2.2-2.8 |
| | | promoter | |
| | | N-(triethoxysilylpropyl)-O- | 0.37- |
| | | polyethylene oxide urethane | 0.45 |
| | | adhesion promoter | |
| | | Optional: Docosanol or | |
| | | acyclovir can be included at | |
| | | commonly applied | |
| | | concentrations | |

### EXAMPLE 1

The patient had a 2.5 day healing time.

1. Use of electromagnetic radiation centred around 1072 nm in combination with a polymerizable composition for the treatment of skin lesions selected from the group comprising herpes virus infections, ulcerations and wounds.

2. Use according to claim 1 wherein the electromagnetic radiation is divergent light between 10° to 50°.

3. Use according to either preceding claim wherein the electromagnetic radiation comprises a bandwidth of between 10 nm to 120 nm.

4. Use according to any preceding claim wherein the electromagnetic radiation is continuous or pulsed.

5. Use according to any preceding claim wherein when the electromagnetic radiation is continuous the intensity is at least 500 μWatts/cm² and up to 500 mWatts/cm².

6. Use according to any one of claims 1 to 4 wherein when the electromagnetic radiation is pulsed the intensity is at least 500 μWatts/cm² peak power and the average power is up to 500 mWatts/cm².

7. Use according to claim 6 wherein when the electromagnetic radiation average power of intensity is in the region of 50-100 mWatts/cm².

8. Use according to either claim 6 or 7 wherein the pulsed electromagnetic radiation it is applied for periods of at least 10-15 μseconds.

9. Use according to any one of claims 6 to 8 wherein frequency/repetition rate is in the range 300-900 Hz.

10. Use according to any preceding claim wherein the electromagnetic radiation is applied to an affected area for at least 3 to 20 minutes.

11. Use according to any preceding claim wherein the electromagnetic radiation is applied to the wound site at least one a day over a period of days, weeks or months until the lesion has healed.

12. Use according to any preceding claim wherein the electromagnetic radiation is applied 1, 2, 3 4, 5 or 6 or more times per day.

13. Use according to any preceding claim wherein the electromagnetic radiation source is a light emitting diode.

14. Use according to claim 13 wherein the radiation source emitter includes a PN junction arranged to emit radiation with a wavelength centring at or about 1072 nm.

15. A method of treatment of a skin lesion, comprising:
applying a polymerizable composition to the affected area of skin;
wherein the polymerizable composition is formulated to cure to a hardness within 5 minutes;
allowing the polymerizable composition to harden to the hardness of between 0 on the Shore 00 durometer scale and 40 on the Shore A durometer scale to so form an elastic protective barrier; and
applying electromagnetic radiation centred around 1072 nm over the cured composition.

16. A method according to claim 15 wherein the skin lesion is selected from the group comprising herpetic sores, diabetic ulcers, pressure sores and wounds.

17. A kit for treating a skin lesion comprising:
(i) a base component comprising at least one of an adhesion promoter, a catalyst that accelerates polymerization of the polymerizable composition;
(ii) a polymerizable composition formulated to polymerize to form a removable seal; and
(iii) a device for emitting electromagnetic radiation centred around 1072 nm.

18. The methods of any of claims 15 to 16 or the kit according to claim 17 further including any one or more of the features recited in claims 2 to 14.
